(19) United States
(12) Patent Application Publication
(10) Pub. No.: US 2022/0218911 A1
(43) Pub. Date: Jul. 14, 2022

(54) DUAL CHAMBER DRUG RESERVOIR AND STOPPER THEREFOR

(71) Applicant: Novo Nordisk A/S, Bagsvaerd (DK)

(72) Inventors: Knud Skifter Winther, Snekkersten (DK); Anders Hammelev Marstrand, Koebenhavn N (DK); Klaus Bendix, Smoerum (DK)

(21) Appl. No.: 17/614,077

(22) PCT Filed: Jun. 4, 2020
(86) PCT No.: PCT/EP2020/065524

§ 371 Date: Nov. 24, 2021

(30) Foreign Application Priority Data
Jun. 7, 2019 (EP) 19179107.8

(51) Int. Cl. A61M 5/315; A61M 5/19

(52) U.S. Cl. CPC A61M 5/31505; A61M 2005/3103; A61M 5/19

(57) ABSTRACT

The present invention concerns an elastomeric stopper (18, 28, 38) for a drug reservoir, comprising: a stopper body (18.1, 28.1, 38.1) extending along a reference axis between a leading end surface (18.2, 28.2, 38.2) and a trailing end surface (18.3, 28.3, 38.3) and having a generally cylindrical sidewall with an outer body diameter, a circumferential convex rib (18.6, 28.6, 38.6) extending radially outwardly from the sidewall, a non-convex trim edge portion (18.7, 28.7, 38.7) at the trailing end surface (18.3, 28.3, 38.3), the non-convex trim edge portion (18.7, 28.7, 38.7) having an outer trim edge diameter which is greater than the outer body diameter and being axially spaced apart from the circumferential convex rib (18.6, 28.6, 38.6), providing an annular recess (18.9, 28.9, 38.9) therebetween. The elastomeric stopper (18, 28, 38) further comprises a channel structure (18.10, 28.10, 38.10) enabling fluid flow from the trailing end surface (18.3, 28.3, 38.3) to the annular recess (18.9, 28.9, 38.9) through the non-convex trim edge portion (18.7, 28.7, 38.7).

### FIELD OF THE INVENTION

The present invention relates to dual chamber reservoirs used in medical injection therapy and to elastomeric stoppers for such reservoirs.

### BACKGROUND OF THE INVENTION

Within some medical treatment areas a combination therapy involving co-administration of at least two drugs is advantageous because of synergistic or additive effects. For example, within diabetes care, in the management of type 2 diabetes mellitus, concomitant use of certain insulin and glp-1 products has been shown to reduce HbA_{1c} levels in subjects, thereby improving glycaemic control.

Many drugs must be administered parenterally to be effective in the body and some of these, e.g. insulin and glp-1, may require one or more doses to be delivered subcutaneously on a daily basis. Subcutaneous drug delivery is often associated with discomfort as many people dislike the thought of having an injection needle inserted through the skin. An undisclosed number of people even suffer from needle-phobia, and these people have a particularly strong desire to escape multiple daily injection therapy. One attractive scenario, therefore, is to reduce the number of required skin penetrations by administering the drugs at the same time, or substantially the same time, through a single injection needle. In some cases, this is achievable by co-formulation of the active ingredients, where the co-formulated product is administered using a conventional injection device. In other cases, e.g. if the active ingredients are unsuitable for co-formulation, the individual substances are stored in separate chambers of a dual chamber, or multi-chamber, reservoir device from which they can be expressed, simultaneously or sequentially, through a single injection needle by use of dedicated expressing means.

U.S. Pat. No. 4,394,863 (Survival Technology, Inc.) discloses an example of a dual chamber reservoir device in the form of an automatic injector with a cartridge having a fixedly mounted hypodermic needle.

EP 1 972 355 A1 (Schott AG) discloses another example of a dual chamber reservoir device in the form of a drug mixing and delivery syringe with a manually operated piston rod and a distal end adapted for reception of an attachable injection needle.

In a pre-use state of both dual chamber reservoir devices a rear chamber is defined between a displaceable sealing stopper and a displaceable partitioning stopper, and a front chamber is defined between the partitioning stopper and an outlet end. The rear chamber holds a first substance and the front chamber holds a second substance. Furthermore, the partitioning stopper is arranged proximally of a bypass channel in the reservoir wall. During use, in both dual chamber reservoir devices, the sealing stopper is advanced by movement of a piston rod element, whereby the first substance becomes pressurised and urges the partitioning stopper forward until it reaches the bypass channel, where the first substance flows past the partitioning stopper and into the front chamber, as continued movement of the piston rod element causes a collapse of the rear chamber. Hence, by movement of a single piston rod element both substances are eventually expressed through a single outlet.

One drawback of such dual chamber reservoir devices is the residual substance in the bypass channel following use, which basically represents a wasted volume of drug. Given that the bypass channel cannot be flushed after collapse of the rear chamber it is desirable to minimise this dead space in the device by designing the bypass channel as small as possible. An important parameter in that respect is the radial height of the bypass channel, which ideally would be minimised.

However, conventional stoppers, which include a number of circumferential sealing ribs, are typically formed in batches, moulded in large mats and cut out individually from these mats. This process inherently leaves a trim edge on each stopper, which is structurally significantly different from the sealing ribs and which potentially poses a problem when the stopper is used with a low bypass channel, because the trim edge is easily deflectable, and once the substance in the rear chamber becomes pressurised the pressure resultantly applied to the rear surface of the partitioning stopper can cause the trim edge to deform into the bypass channel and prevent fluid flow therethrough. In contrast to the sealing ribs, which are respectively structured to seal against an interior annular surface of the reservoir wall, the trim edge can thus end up undesirably sealing the bypass channel.

### SUMMARY OF THE INVENTION

It is an object of the invention to eliminate or reduce at least one drawback of the prior art, or to provide a useful alternative to prior art solutions.

In particular, it is an object of the invention to provide a solution whereby batch produced stoppers can be used in dual chamber reservoir based drug delivery devices with low bypass channels without a risk of device malfunctioning.

It is accordingly a further object of the invention to provide a dual chamber drug reservoir device with a bypass channel, which can employ stoppers with trim edges regardless of the bypass channel height.

It is an even further object of the invention to provide a stopper suitable for use in a dual chamber drug reservoir device having a low bypass channel.

It is an even further object of the invention to provide a dual chamber drug delivery device with a bypass channel, which offers a fail-safe transfer of liquid substance from the rear chamber to the front chamber while exhibiting a diminutive dead space.

In the disclosure of the present invention, aspects and embodiments will be described which will address one or more of the above objects and/or which will address objects apparent from the following text.

In one aspect the invention provides an elastomeric stopper according to claim 1.

Hence, an elastomeric stopper for a drug reservoir is provided. The elastomeric stopper comprises a stopper body which extends along a reference axis between a leading end surface and a trailing end surface and which has a generally cylindrical sidewall with an outer body diameter. The stopper body comprises a circumferential rib which extends radially outwardly from the sidewall, for sealing interaction with an interior annular wall portion of the drug reservoir, and a circumferential trim edge portion at the trailing end surface. The trim edge portion has an outer trim edge diameter which is greater than the outer body diameter and is axially spaced apart from the circumferential rib. An annular portion of the generally cylindrical sidewall thus separates the circumferential rib and the trim edge portion and can be said to provide an annular recess with respect to the two. The circumferential rib is convex and has a relatively high structural resistance to both axial and radial deformation, which is ideal for providing the intended sealing against the interior annular wall portion of the drug reservoir, whereas the trim edge portion, being an unintended by-product of the stopper manufacturing process, is non-convex, e.g. concave, and easily deformable.

The elastomeric stopper further comprises a channel structure enabling fluid flow from the trailing end surface to the annular recess through the trim edge portion. Thereby, a liquid substance can pass through the trim edge portion during use of the elastomeric stopper in e.g. a dual chamber reservoir device, such that even if the trim edge portion deforms into the bypass channel the liquid substance will enter the annular recess between the circumferential rib and the trim edge portion, and since the annular recess is not in sealing contact with the reservoir wall the liquid substance will continue to the bypass channel along the pathway provided between the annular recess and the reservoir wall, driven by the positive pressure in the rear chamber. A fail-safe transfer of the liquid substance from the rear chamber to the front chamber is thus possible even in a dual chamber reservoir based device exhibiting a diminutive dead space in the form of a low bypass channel.

The stopper body may comprise a plurality of axially spaced apart circumferential convex ribs, each circumferential convex rib extending radially outwardly from the sidewall and being adapted to provide a sealing interface to the reservoir wall. In that case one of said plurality of axially spaced apart circumferential convex ribs is a leading rib, being positioned nearest the leading end surface, and another of the plurality of axially spaced apart circumferential convex ribs is a trailing rib, being positioned nearest the trailing end surface, and the annular recess is provided between the trim edge portion and the trailing rib.

In particular embodiments of the invention the channel structure comprises at least one bore through the trim edge portion, the at least one bore having an inlet opening in the trailing end surface and an outlet opening in the annular recess.

In other embodiments of the invention the channel structure comprises at least one peripheral cut-out forming a part-cylindrical indentation in the trim edge portion.

In another aspect the invention provides a drug reservoir comprising an elastomeric stopper as described above. Hence, a drug reservoir may be provided comprising a reservoir body comprising a generally cylindrical inner wall with an axially extending bypass channel therein, a proximal end section, a distal outlet end section, the elastomeric stopper slidably arranged between the distal outlet end section and the proximal end section, and a second stopper slidably arranged between the elastomeric stopper and the proximal end section. A front chamber is thereby defined by the distal outlet end section, a first portion of the reservoir body, and the elastomeric stopper, and a rear chamber is defined by the elastomeric stopper, a second portion of the reservoir body, and the rear stopper. The elastomeric stopper may initially be positioned to provide a fluid tight seal with the generally cylindrical inner wall proximally of the bypass channel. Furthermore, the rear chamber may hold a first substance, e.g. comprising a first liquid volume, and the front chamber may hold a second substance, e.g. comprising a second liquid volume or a dry powder.

Due to the channel structure in the elastomeric stopper when the rear chamber is pressurised by distal movement of the rear stopper, e.g. executed by applying a driving force to the rear stopper using a dedicated stopper actuator, and the first substance resultantly exerts a pressure onto the trailing end surface of the elastomeric stopper, it doesn't matter if the trim edge portion deforms into the bypass channel because a flow path to the annular recess through the trim edge portion will always be open, thus ensuring passage of the first substance to the front chamber through the bypass channel regardless of the height of the bypass channel. A dual chamber drug reservoir with a minimised bypass channel, exhibiting a diminutive dead space, can accordingly be utilised without risking a malfunction during fluid transfer from the rear chamber to the front chamber.

The drug reservoir may be a syringe, e.g. comprising a Luer fitting or a staked needle at the distal outlet end section. In case of the latter the drug reservoir further comprises an injection needle with a lumen, wherein the lumen is in fluid communication with the distal outlet end section. Alternatively, the drug reservoir may be a cartridge type reservoir, in which case the distal outlet end section is closed by a penetrable self-sealing septum.

In a further aspect the invention provides a drug delivery device comprising a drug reservoir as described above in combination with a drug expelling mechanism. The drug expelling mechanism may comprise a plunger rod operable to displace the rear stopper relative to the generally cylindrical wall, i.e. operable to pressurise the rear chamber and thereby execute a transfer of the first substance to the front chamber. The plunger rod may be configured for manual operation by the user, or it may form part of an automatic drug expelling mechanism and be configured for advancement into the drug reservoir upon release of an energy storage means, such as a spring member or a compressed gas.

For the avoidance of any doubt, in the present context the term “drug” designates a medium which is used in the treatment, prevention or diagnosis of a condition, i.e. including a medium having a therapeutic or metabolic effect in the body. Further, the terms “distal” and “proximal” denote positions at, or directions along, a drug delivery device, a drug reservoir, or a needle unit, where “distal” refers to the drug outlet end and “proximal” refers to the end opposite the drug outlet end.

In the present specification, reference to a certain aspect or a certain embodiment (e.g. “an aspect”, “a first aspect”, “one embodiment”, “an exemplary embodiment”, or the like) signifies that a particular feature, structure, or characteristic described in connection with the respective aspect or embodiment is included in, or inherent of, at least that one aspect or embodiment of the invention, but not necessarily in/of all aspects or embodiments of the invention. It is emphasized, however, that any combination of the various features, structures and/or characteristics described in relation to the invention is encompassed by the invention unless expressly stated herein or clearly contradicted by context.

The use of any and all examples, or exemplary language (e.g., such as, etc.), in the text is intended to merely illuminate the invention and does not pose a limitation on the scope of the same, unless otherwise claimed. Further, no language or wording in the specification should be construed as indicating any non-claimed element as essential to the practice of the invention.

### BRIEF DESCRIPTION OF THE DRAWINGS

In the following the invention will be further described with references to the drawings, wherein

FIG. 1a is a longitudinal section view of a dual chamber syringe according to the prior art,

FIG. 1b is a close-up view of a section of the syringe of FIG. 1a,

FIG. 2 is a longitudinal section view of a conventional type stopper used in dual chamber syringe devices,

FIG. 3 is a longitudinal section view of the stopper of FIG. 2 in a syringe with a low bypass channel,

FIGS. 4a and 4b are different views of a stopper according to a first embodiment of the invention,

FIGS. 5a and 5b are different views of a stopper according to a second embodiment of the invention, and

FIGS. 6a and 6b are different views of a stopper according to a third embodiment of the invention.

In the figures like structures are mainly identified by like reference numerals.

### DESCRIPTION OF EXEMPLARY EMBODIMENTS

When/If relative expressions, such as “upper” and “lower”, “left” and “right”, “horizontal” and “vertical”, “clockwise” and “counter-clockwise”, etc., are used in the following, these refer to the appended figures and not necessarily to an actual situation of use. The shown figures are schematic representations for which reason the configuration of the different structures as well as their relative dimensions are intended to serve illustrative purposes only.

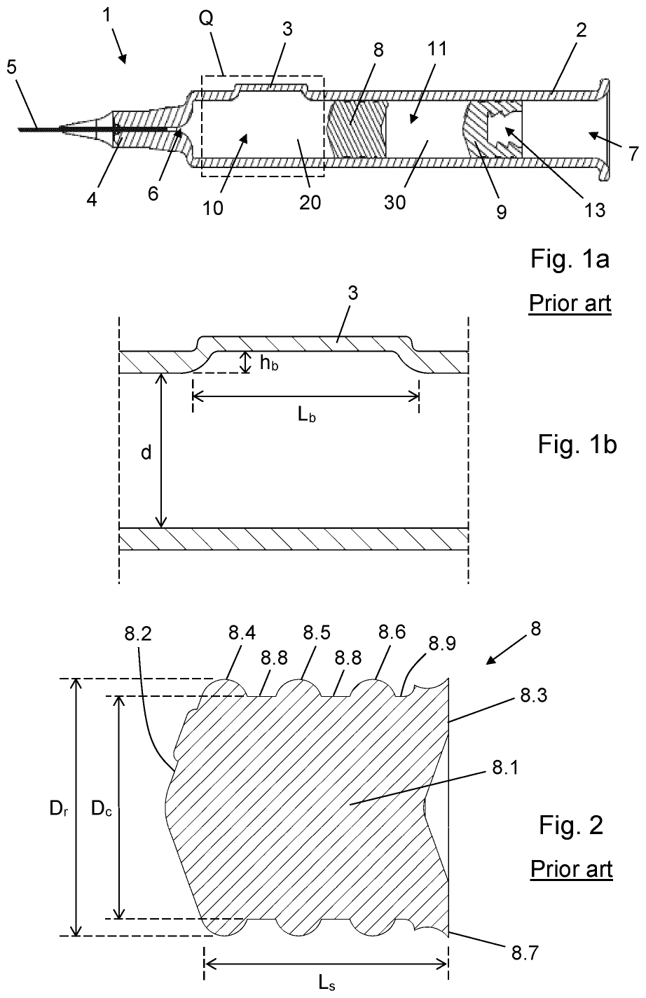

FIG. 1a is a longitudinal section view of a conventional prior art dual chamber syringe 1 in a pre-use state, i.e. in a state as supplied by the manufacturer (albeit without a rigid needle protector), and FIG. 1b is an enlargement of a section Q thereof.

The syringe 1 comprises a generally cylindrical syringe barrel 2 with a bypass channel 3 and a narrowed distal end portion 4. An injection needle 5 is fixed to the distal end portion 4 and establishes fluid communication to an outlet portion 6. A front stopper 8 is arranged in the syringe barrel 2 between the outlet portion 6 and an open proximal end 7, and a front chamber 10 is thereby defined by the outlet portion 6, a front portion of the syringe barrel 2 comprising the bypass channel 3, and the front stopper 8. A rear stopper 9 is arranged in the syringe barrel 2 between the front stopper 8 and the open proximal end 7, and a rear chamber 11 is thereby defined by the front stopper 8, a middle portion of the syringe barrel 2, and the rear stopper 9. The rear stopper 9 has a cavity 13 adapted to receive an end portion of a plunger rod (not shown), which is then actuatable to advance the rear stopper 9 through the syringe barrel 2. The front chamber 10 holds a first liquid substance 20, and the rear chamber 11 holds a second liquid substance 30.

As can be seen from FIG. 1b the bypass channel 3 is defined by a bypass length L_{b} and a bypass height h_{b}. The generally cylindrical syringe barrel 2 has an inner diameter d. However, at the bypass channel 3 the inner diameter is accordingly d+h_{b}.

FIG. 2 is a longitudinal section view of the front stopper 8 which comprises a stopper body 8.1 extending along a longitudinal stopper axis and having a leading end face 8.2 and a trailing end face 8.3. The stopper body 8.1 further has three convex circumferential sealing ribs, a leading rib 8.4, an intermediate rib 8.5, and a trailing rib 8.6, as well as a non-convex trim edge 8.7. The three circumferential sealing ribs are axially spaced apart by respective annular recesses 8.8 and serve to seal against an interior wall of the syringe barrel 2. Hence, the transversal dimension of the front stopper 8 varies along the longitudinal stopper axis between a rib diameter Dᵣ and a stopper core diameter D_{c}. The trim edge 8.7, which serves no functional purpose but is inherently present due to the separation of the stopper body 8.1 from a batch rubber sheet, is axially spaced apart from the trailing rib 8.6 by a trailing annular recess 8.9. In the present example the trim edge 8.7 has a maximum diameter approximately of the size of the rib diameter D₁, and a potential sealing length, Lₛ, of the front stopper 8 is thus defined as the axial distance between a leading curved portion of the leading rib 8.4 and a trailing face of the trim edge 8.7.

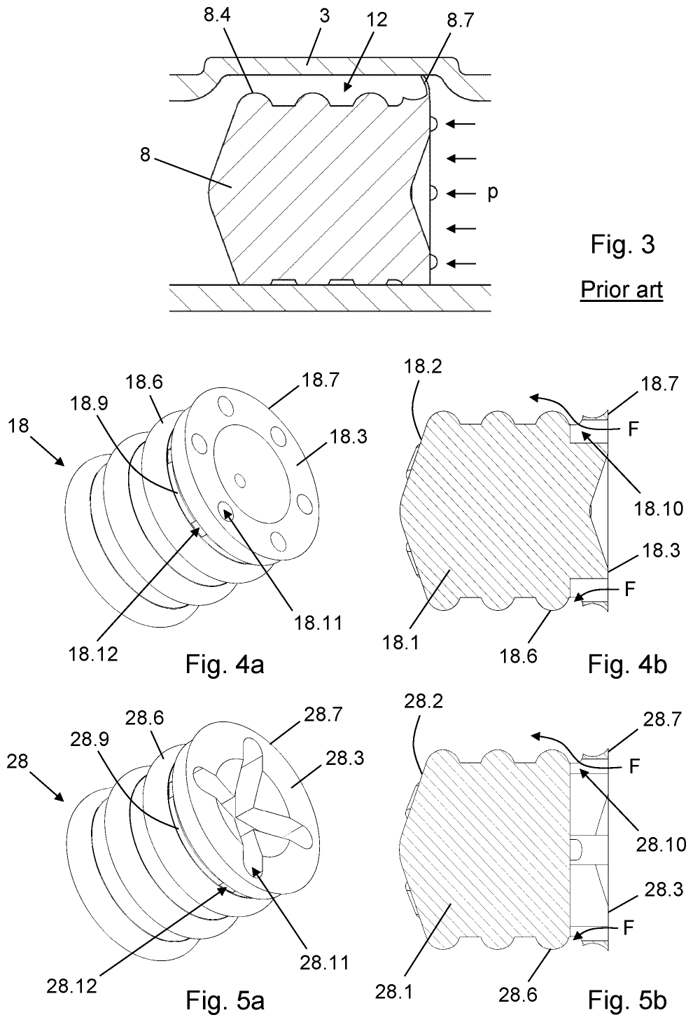

A design prerequisite for liquid flow past the front stopper 8 is that Lₛ<L_{b}. However, as illustrated in FIG. 3, which sketches a version of the syringe 1 where the bypass channel 3 has a low bypass height h_{b}, when the second liquid substance 30 is pressurised due to an advancement of the rear stopper 9 a pressure p is resultantly exerted onto the trailing end face 8.3 which at first causes the front stopper 8 to advance towards a position within the bypass channel 3, but which when the trailing end face 8.3 reaches the bypass channel 3 may cause the trim edge 8.7 to deform thereinto and prevent the second liquid substance 30 from entering an otherwise established flow passage 12 between the exterior of the front stopper 8 and the syringe barrel 2 (the bypass channel 3 has a limited circumferential extent which prevents any of the three circumferential sealing ribs from entering, but the trim edge 8.7 is a much more easily deformable structure). The desired transfer of the second liquid substance 30 to the front chamber 10 can thus not be realised, leading to a malfunction of the syringe 1, as only the first liquid substance 20 is then administrable.

This problem can be overcome by employing a specially designed front stopper where liquid flow into the passage 12 is guaranteed even if the trim edge is deformed into the bypass channel 3.

One such is shown in FIG. 4, where FIG. 4a is a perspective view and FIG. 4b a longitudinal section view of a front stopper 18 according to a first exemplary embodiment of the invention. In FIG. 4a the front stopper 18 is depicted from a proximal perspective, i.e. seen from a trailing end, and like the conventional front stopper 8, which the front stopper 18 is supposed to replace, it comprises a stopper body 18.1 extending along a longitudinal stopper axis and having a leading end face 18.2, a trailing end face 18.3, three axially spaced convex circumferential sealing ribs including a trailing rib 18.6, and a non-convex trim edge 18.7. A trailing annular recess 18.9 is present between the trailing rib 18.6 and the trim edge 18.7.

The inventive idea is to enable liquid flow to the trailing annular recess 18.9 through the trim edge 18.7. To this end a plurality of channels 18.10 (FIG. 4b) have been established by penetration of the trim edge 18.7. A plurality of inlet openings 18.11 in fluid communication with a corresponding plurality of outlet openings 18.12 are thereby provided, and when the second liquid substance 30 becomes pressurised a volume thereof will flow through the channels 18.10 and into the trailing annular recess 18.9.

As long as the trailing rib 18.6 is in sealing contact with the syringe barrel 2 proximally of the bypass channel 3 the second liquid substance 30 will apply a driving force to the front stopper 8, advancing the front stopper 8 distally in the syringe barrel 2. However, as soon as the trailing rib 18.6 enters the bypass channel 3 the pressure in the rear chamber 11 will force the liquid in the trailing annular recess 18.9 into the flow passage 12 between the stopper exterior and the syringe barrel 2. At this point the front stopper 18 will stop advancing and as the rear stopper 9 continues to advance the second liquid substance 30 is forced through the channels 18.10 into the trailing annular recess 18.9 and from there on into and through the flow passage 12, as indicated by flow arrows F.

The transfer of the second liquid substance 30 to the front chamber 10 continues until the rear chamber 11 eventually is completely collapsed, at which point the rear stopper 9 abuts the front stopper 8 and further advancement of the rear stopper 9 by means of the plunger rod causes a gradual collapse of the front chamber 10, all expellable liquid thereby being forced through the outlet portion 6 and the injection needle 5.

Hence, the presence of the channels 18.10 through the trim edge 18.7 ensures a path from the rear chamber 11 to the flow passage 12 which the second liquid substance 30 can flow through, regardless of the size and deformability of the trim edge 18.7. A fail-safe liquid transfer from the rear chamber 11 to the front chamber 10 is thereby provided, even in a syringe with a minimised bypass height h_{b}.

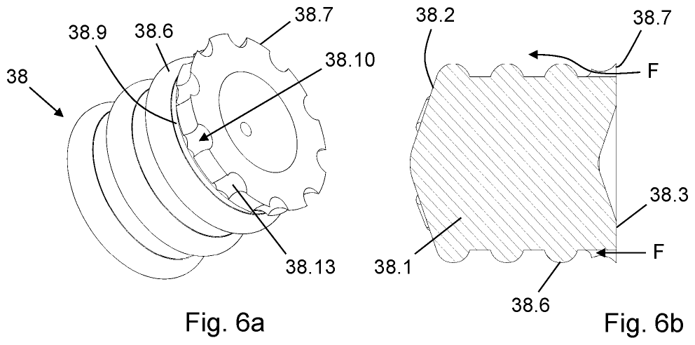

The same basic principle is utilised in two alternative embodiments shown, respectively, in FIGS. 5 and 6.

FIG. 5a is a perspective proximal view, and FIG. 5b a longitudinal section view, of a front stopper 28 according to a second embodiment of the invention. Like the embodiment of FIG. 4 the front stopper 28 comprises a stopper body 28.1 extending along a longitudinal stopper axis and having a leading end face 28.2, a trailing end face 28.3, three axially spaced convex circumferential sealing ribs including a trailing rib 28.6, and a non-convex trim edge 28.7. A trailing annular recess 28.9 is present between the trailing rib 28.6 and the trim edge 28.7, and a plurality of channels 28.10 (FIG. 5b) have been established by penetration of the trim edge 28.7. A plurality of inlet openings 28.11 in fluid communication with a corresponding plurality of outlet openings 28.12 are thereby provided, and when the second liquid substance 30 becomes pressurised a volume thereof will flow through the channels 28.10 and into the trailing annular recess 28.9. The working principle is the same as that described above with respect to the front stopper 18 according to the first embodiment of the invention, the only significant difference from the first embodiment, apart from the number of inlet openings and outlet openings, being the establishment of the channels 28.10. The tool used to establish the channels 28.10 has a geometric cross configuration and is thus more robust when penetrating the trim edge 28.7 than for example six separate thin sticks used to penetrate the trim edge 18.7 in the establishment of the channels 18.10 in the front stopper 18.

FIG. 6a is a perspective proximal view, and FIG. 6b a longitudinal section view, of a front stopper 38 according to a third embodiment of the invention. Like the previous embodiments the front stopper 38 comprises a stopper body 38.1 extending along a longitudinal stopper axis and having a leading end face 38.2, a trailing end face 38.3, three axially spaced convex circumferential sealing ribs including a trailing rib 38.6, and a non-convex trim edge 38.7. A trailing annular recess 38.9 is present between the trailing rib 38.6 and the trim edge 38.7, and a plurality of circumferentially spaced apart channels 38.10 (FIG. 5b) have been established as semi-cylindrical cut-outs 38.13 in the periphery of the trim edge 38.7. This ensures that the rim of the trim edge 38.7 cannot seal completely against the syringe barrel 2 and that the second liquid substance 30 accordingly can flow through the trim edge 38.7 between the rim of the trim edge 38.7 and the syringe barrel 2 and enter the trailing annular recess 38.9 from where it can pass to the flow passage 12 when the trailing annular recess 38.9 reaches the bypass channel 3.

It is noted that even though each of the specifically disclosed embodiments of the invention has three circumferential sealing ribs it is understood that the exact number of such ribs is irrelevant to the inventive concept of establishing a permanent flow path through the trim edge to the trailing annular recess. This solution is thus equally applicable to other types of stoppers having, for example, one, two or four circumferential sealing ribs.

1. An elastomeric stopper for a drug reservoir, comprising:
a stopper body extending along a reference axis between a leading end surface and a trailing end surface and having a generally cylindrical sidewall with an outer body diameter,
a circumferential convex rib extending radially outwardly from the sidewall,
a non-convex trim edge portion at the trailing end surface, the non-convex trim edge portion having an outer trim edge diameter which is greater than the outer body diameter and being axially spaced apart from the circumferential convex rib, providing an annular recess therebetween,
further comprising a channel structure enabling fluid flow from the trailing end surface to the annular recess through the non-convex trim edge portion.

2. The elastomeric stopper according to claim 1, wherein the channel structure comprises at least one bore through the non-convex trim edge portion, the at least one bore having an inlet opening in the trailing end surface and an outlet opening in the annular recess.

3. The elastomeric stopper according to claim 1, wherein the channel structure comprises at least one peripheral cut-out forming a part-cylindrical indentation in the non-convex trim edge portion.

4. A drug reservoir comprising:
a reservoir body comprising a generally cylindrical wall with an axially extending bypass channel therein,
a proximal end section,
a distal outlet end section,
an elastomeric stopper according to any of the preceding claims slidably arranged between the distal outlet end section and the proximal end section and initially providing a fluid tight seal with the generally circular cylindrical wall proximally of the bypass channel, a front chamber thereby being defined by the distal outlet end section, a first portion of the reservoir body, and the elastomeric stopper, and
a rear stopper slidably arranged between the elastomeric stopper and the proximal end section, a rear chamber thereby being defined by the elastomeric stopper, a second portion of the reservoir body, and the rear stopper.

5. The drug reservoir according to claim 4, further comprising an injection needle with a lumen, the lumen being in fluid communication with the distal outlet end section.

6. The drug reservoir according to claim 4, wherein the distal outlet end section is closed by a penetrable self-sealing septum.

7. A drug delivery device comprising a drug reservoir according to claim 4 together with a drug expelling mechanism, wherein the drug expelling mechanism comprises a plunger rod operable to displace the rear stopper relative to the generally cylindrical wall.
